(19) United States
(12) Patent Application Publication
(10) Pub. No.: US 2019/0246922 A1
(43) Pub. Date: Aug. 15, 2019

(54) BIOLOGICAL INFORMATION MEASUREMENT DEVICE

(71) Applicant: SEIKO EPSON CORPORATION, Tokyo (JP)

(72) Inventors: Atsushi MATSUO, Azumino-shi (JP); Kazuyuki KANO, Suwa-shi (JP); Yoshiaki MATSUMOTO, Ina-shi (JP); Hiroki KOBAYASHI, Hara-mura (JP)

(73) Assignee: SEIKO EPSON CORPORATION, Tokyo (JP)

(21) Appl. No.: 16/276,126

(22) Filed: Feb. 14, 2019

(30) Foreign Application Priority Data
Feb. 15, 2018 (JP) 2018-024988

(51) Int. Cl. A61B 5/024

(52) U.S. Cl. CPC A61B 5/02427; A61B 2562/146; A61B 2562/0238; A61B 5/02438

(57) ABSTRACT

A biological information measurement device adapted to measure biological information of a living body includes a board having a first surface and a second surface adapted to form two surfaces of the board with the first surface, a circuit element disposed on the first surface, and adapted to execute a predetermined process, and a sensor section disposed on the second surface, and adapted to detect the biological information, and the sensor section has a light emitter adapted to irradiate the living body with light, a light receiver adapted to receive reflected light reflected by the living body, and a sealing material having a light transmissive property, having contact with the living body, and adapted to seal the light emitter and the light receiver on the board.

### CROSS-REFERENCES TO RELATED APPLICATIONS

This application claims priority to Japanese Patent Application No. 2018-024988, filed on Feb. 15, 2018, the entirety of which is herein incorporated by reference.

### BACKGROUND

### 1. Technical Field

The present invention relates to a biological information measurement device.

### 2. Related Art

In the past, there has been known a biological information measurement device which is attached to a living body to measure the biological information. As such a biological information measurement device, there is known a pulse wave sensor for measuring a pulse wave (see, e.g., JP-A-2012-120772 (Document 1)).

The pulse wave sensor described in Document 1 has a configuration shaped like a bracelet which is mounted on the wrist to measure the pulse wave, and is provided with a measurement unit, a power unit and a communication unit, a cable for electrically connecting these units to each other, a bracelet-shaped housing and a display unit.

The measurement unit is a unit for measuring the pulse wave, and is housed in the bracelet-shaped housing. The measurement unit has a board, a photosensor directly mounted on the obverse surface of the board, a measurement window, and an amplifier circuit and an arithmetic circuit directly mounted on the reverse surface of the board.

The photosensor has a light emitter and a light receiver. The wrist is irradiated with the light emitted from the light emitter via the measurement window as a light transmissive member, and the reflected light returning from the wrist through the living body is received by the light receiver via the measurement window, and the light receiver detects the intensity of the light to thereby obtain the pulse wave data. The pulse wave data is input to the arithmetic circuit through the amplifier circuit, and a variety of types of information (fluctuation of the pulse wave, a heart rate, heart-rate variability and an acceleration pulse wave) related to the pulse wave are obtained by the arithmetic circuit.

Here, in the pulse wave sensor described in Document 1, since the measurement window as the light transmissive member is disposed separately from the photosensor, the distance between the photosensor and the living body (the wrist) is apt to increase as much as the thickness of the measurement window. If the distance between the photosensor and the living body increases as described above, attenuation of the light emitted from the light emitter and attenuation of the light received by the light receiver become apt to occur, and there is a problem that the detection accuracy of the pulse wave deteriorates.

### SUMMARY

An advantage of some aspects of the invention is to provide a biological information measurement device capable of improving the measurement accuracy of the biological information.

A biological information measurement device according to an aspect of the invention is a biological information measurement device adapted to measure biological information of a living body including a board having a first surface and a second surface adapted to form two surfaces of the board with the first surface, a circuit element disposed on the first surface, and adapted to execute a predetermined process, and a sensor section disposed on the second surface, and adapted to detect the biological information, wherein the sensor section has a light emitter adapted to irradiate the living body with light, a light receiver adapted to receive reflected light reflected by the living body, and a sealing material having a light transmissive property, having contact with the living body, and adapted to seal the light emitter and the light receiver on the board.

It should be noted that sealing denotes that the sealing target is enclosed by a sealing material inside, but in the present specification, sealing is not limited to completely enclosing the sealing target inside, but includes the case in which a part of the sealing target projects outside.

According to such a configuration, on the second surface forming the two surfaces of the board together with the first surface on which the circuit element is disposed, there are disposed the light emitter and the light receiver, and in addition, there is disposed the sealing material for sealing the light emitter and the light receiver on the board. According to this configuration, it is possible to protect the light emitter and the light receiver without disposing the measurement window as the light transmissive member as in the case of the pulse wave sensor described in Document 1 described above.

Further, the sealing material for sealing the light emitter and the light receiver has contact with the living body. According to this configuration, it is possible to shorten the distance from the light emitter to the living body and the distance from the living body to the light receiver compared to the configuration in which the light transmissive member is separately provided from the photosensor as in the pulse wave sensor described in Document 1 described above. Therefore, it is possible to suppress the attenuation of the light with which the living body is irradiated by the light emitter, and the attenuation of the light entering the light receiver from the living body, and thus, it is possible to suppress the reduction of the received light intensity in the light receiver. Therefore, it is possible to improve the detection accuracy of the biological information, and in addition, it is possible to achieve thickness reduction compared to the pulse wave sensor described in Document 1 having the measurement window.

In the aspect of the invention described above, it is preferable that the circuit element includes a processing circuit adapted to determine the biological information based on a signal output from the light receiver.

According to such a configuration, the function as the biological information measurement device can be realized by the configuration provided to the board. Therefore, since there is no need for separately providing the board provided with the light emitter and the light receiver and the board provided with the processing circuit, the configuration of the biological information measurement device can be simplified, and in addition, it is possible to achieve further reduction in size of the biological information measurement device.

In the aspect of the invention described above, it is preferable that at least either one of the light emitter and the light receiver is constituted by a bare chip.

Here, the bare chip denotes an element not packaged. For example, as the bare chip constituting the light emitter, there can be cited a light emitting diode (LED) element not packaged and an organic electro-luminescence (EL) element not packaged. Further, for example, as the bare chip constituting the light receiver, there can be cited a photodiode (PD) element not packaged. Further, the description that either one of the light emitter and the light receiver is the bare chip includes the case in which only the light emitter is the bare chip, the case in which only the light receiver is the bare chip, and the case in which both of the light emitter and the light receiver are the bare chips.

According to such a configuration, since the bare chip is smaller in thickness dimension (the rising dimension from the board in the case of disposing the chip on the board) than the packaged chip, it is possible to reduce the thickness dimension of the sealing material, and by extension, to reduce the thickness dimension of the biological information measurement device. Therefore, it is possible to further reduce the thickness of the biological information measurement device.

In the aspect of the invention described above, it is preferable that the sensor section has a light blocking wall disposed between the light emitter and the light receiver, and the light blocking wall is sealed by the sealing material together with the light emitter and the light receiver.

It should be noted that in this configuration, providing the light emitter and the light receiver are enclosed in the sealing material, a part of the light blocking wall can slightly project outside the sealing material.

According to such a configuration, the light directly proceeding from the light emitter toward the light receiver without the intervention of the living body can be blocked by the light blocking wall. Therefore, it is possible to improve the detection accuracy and the measurement accuracy of the biological information.

Further, since the light blocking wall is sealed by the sealing material together with the light emitter and the light receiver, there is no need for separately disposing the light blocking wall outside the sealing material. Therefore, it is possible to achieve the reduction in thickness of a biological information detection sensor in combination with the fact that it is possible to shorten the distance between the contact surface with the living body in the sealing material and the light emitter and the distance between the contact surface with the living body in the sealing material and the light receiver.

In the aspect of the invention described above, it is preferable that the light emitter has a first light emitter and a second light emitter, the light receiver is disposed between the first light emitter and the second light emitter, and the light blocking wall is disposed between the first light emitter and the light receiver and between the second light emitter and the light receiver.

According to such a configuration, by using the light emitter at the position suitable for the detection of the biological information out of the first light emitter and the second light emitter, it is possible to improve the detection accuracy of the biological information. In contrast, since it is possible to increase the received light intensity in the light receiver by using each of the first light emitter and the second light emitter, even in this case, it is possible to improve the detection accuracy of the biological information.

Further, due to the light blocking wall, it is possible to block the light directly proceeding from the first light emitter toward the light receiver and the light directly proceeding from the second light emitter toward the light receiver. Therefore, since it is possible to prevent the light having not passed through the living body from entering the light receiver, it is possible to further improve the detection accuracy of the biological information.

In the aspect of the invention described above, it is preferable that the light blocking wall surrounds the light receiver viewed from a direction perpendicular to the board.

Here, since the light emitter and the light receiver are sealed by the sealing material, there is a possibility that a part of the light emitted from the light emitter proceeds inside the sealing material while repeating the internal reflection, and then enters the light receiver from a different side from the light emitter side in the light receiver.

In contrast, according to the configuration described above, it is possible not only to block the light emitted from each of the light emitter and then proceeding inside the sealing material without the intervention of the living body toward the light receiver with the light blocking wall, but also to block the disturbance light entering the sealing material and then proceeding toward the light receiver with the light blocking wall. Therefore, it is possible to further improve the detection accuracy of the biological information.

In the aspect of the invention described above, it is preferable that a position of a tip part on a projecting direction side from the board in the light blocking wall roughly coincides with a position of a surface from which the light emitted from the light emitter is emitted to an outside in the sealing material.

It should be noted that the surface from which the light emitted from the light emitter is emitted to the outside in the sealing material is a contact surface capable of having contact with the living body, and in other words, a surface which is located between the light emitter and the light receiver in the sealing material, from which the light emitted from the light emitter is emitted toward the living body, and which the light reflected by the living body enters. This surface is hereinafter defined as an incident/emission surface in the sealing material.

Here, in the case in which the light emitter emits the light with a predetermined emission angle, there is a possibility that a part of the light emitted from each of the light emitter is internally reflected by the incident/emission surface described above. In such a case, if the tip part in the light blocking wall is located on the board side from the incident/emission surface described above, there arises a possibility that the part of the light described above enters the light receiver beyond the light blocking wall.

To cope with the above, since the position of the tip part described above in the light blocking wall roughly coincides with the position of the incident/emission surface described above, it is possible to block the light internally reflected by the incident/emission surface described above with the light blocking wall, and thus, it is possible to prevent the light internally reflected from entering the light receiver. Therefore, the deterioration of the detection accuracy of the biological information can be suppressed.

In the aspect of the invention described above, it is preferable that a surface from which the light emitted from the light emitter is emitted to an outside in the sealing material is a curved surface.

According to such a configuration, by providing the surface described above in the sealing material with a lens shape, it is possible to provide a lens function to the sealing material. Therefore, by using, for example, a convexly curved surface or a concavely curved surface as the surface described above in the sealing material, it is possible to emit the light emitted from the light emitter to the living body so as to be diffused or converged. Further, for example, by using the concavely curved surface as the surface described above in the sealing material, it is possible to converge the light reflected by the living body on the light receiver. As described above, it is possible to effectively irradiate the living body with the light emitted from the light emitter in accordance with the purpose of the biological information measurement device, and in addition, it is possible to make the light entering the surface efficiently enter the light receiver. Therefore, it is possible to further improve the detection accuracy of the biological information.

In the aspect of the invention described above, it is preferable that a surface from which the light emitted from the light emitter is emitted to an outside in the sealing material is a flat surface.

Here, the thickness dimension of the sealing material depends on the cross-sectional shape of the surface described above in the sealing material. Therefore, by using a flat surface as the surface described above in the sealing material, it is possible to reduce the thickness dimension of the sealing material, and by extension, to reduce the thickness dimension of the biological information measurement device. Therefore, it is possible to achieve further reduction in thickness of the biological information measurement device.

In the aspect of the invention described above, it is preferable that the biological information measurement device further includes a signal processor disposed on the second surface, and adapted to process a signal output from the light receiver, and a housing adapted to house the board, the housing has a recessed part, and at least a part of the signal processor is disposed inside the recessed part.

It should be noted that as the signal processor, there can be cited an analog front end (AFE) for performing amplification, noise removal and A/D conversion on the signal output from the light receiver.

According to such a configuration, it is possible to reduce the thickness of the housing for housing the board compared to the case in which the recessed part is absent. Therefore, it is possible to achieve further reduction in thickness of the biological information measurement device.

### BRIEF DESCRIPTION OF THE DRAWINGS

The invention will be described with reference to the accompanying drawings, wherein like numbers reference like elements.

FIG. 1 is a front view showing a biological information measurement device according to a first embodiment of the invention.

FIG. 2 is a diagram showing a back side part of the biological information measurement device in the first embodiment described above.

FIG. 3 is a diagram showing an internal configuration of the biological information measurement device in the first embodiment described above.

FIG. 4 is a perspective view showing a rear case and a control section in the first embodiment described above.

FIG. 5 is a perspective view of the control section in the first embodiment described above viewed from a light emission side.

FIG. 6 is a perspective view showing an arrangement of the control section with respect to the rear case in the first embodiment described above.

FIG. 7 is a diagram of the control section in the first embodiment described above viewed from a lateral side.

FIG. 8 is a perspective view showing the rear case in which the control section in the first embodiment described above is disposed.

FIG. 9 is a diagram showing an internal configuration of a biological information measurement device according to a second embodiment of the invention.

FIG. 10 is a side view showing a control section provided to a biological information measurement device according to a third embodiment of the invention.

### DESCRIPTION OF EXEMPLARY EMBODIMENTS

### First Embodiment

A first embodiment of the invention will hereinafter be described based on the accompanying drawings.

### Schematic Configuration of Biological Information Measurement Device

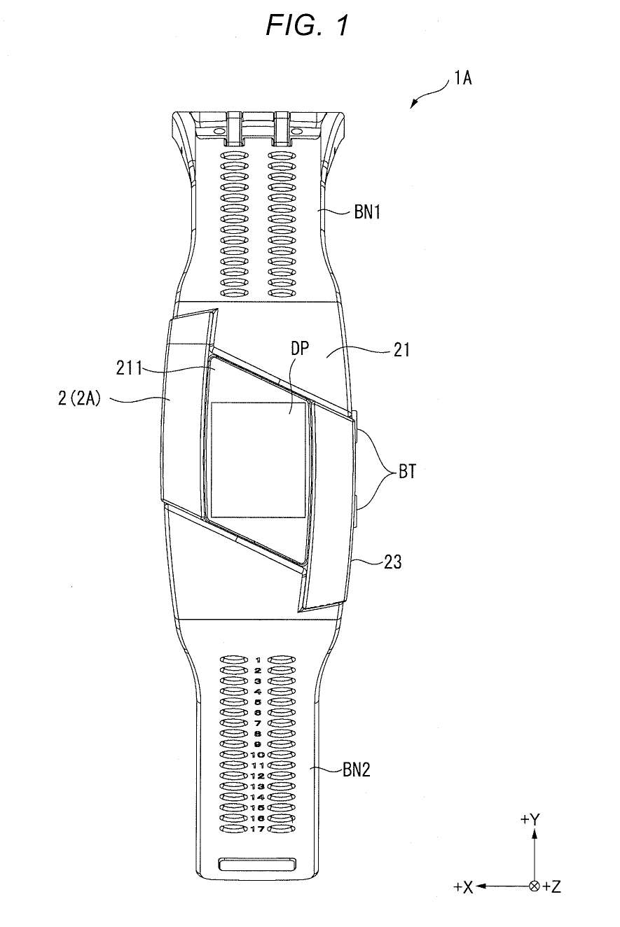

FIG. 1 is a front view showing a biological information measurement device 1A according to the present embodiment.

The biological information measurement device 1A (hereinafter abbreviated as a measurement device 1A in some cases) according to the present embodiment is wearable equipment used while attached to a body of a user, and measures the biological information of the user. Specifically, the measurement device 1A is used while mounted on a mounting target region such as a wrist of the user, detects the pulse wave of the user as a type of the biological information to measure the pulse rate as another type of the biological information. Further, the measurement device 1A has one of the features in the position and the configuration of a sensor section 5A described later.

As shown in FIG. 1, the measurement device 1A is provided with a housing 2 and bands BN1, BN2.

It should be noted that in the following description, a direction from a front side part 21 of the housing 2 toward a rear side part 22 is defined as a +Z direction, and two directions perpendicular to the +Z direction and perpendicular to each other are defined as a +X direction and a +Y direction. Among these directions, +X direction is defined as a 9 o'clock direction viewed from a position opposed to the front side part 21, and the +Y direction is defined as a 12 o'clock direction. Further, although not shown in the drawings, an opposite direction to the +Z direction is defined as a −Z direction. The same applies to a −X direction and a −Y direction. It should be noted that the +Z direction is also a direction in which light emitters 51 constituting the sensor section 5A described later mainly emit the light, and the −Z direction is also a direction in which the light having been reflected by the living body mainly enters a light receiver 52.

The band BN1 extends from an end part on the +Y direction side of the housing 2 toward the +Y direction, and the band BN2 extends from an end part on the −Y direction side of the housing 2 toward the −Y direction. By the bands BN1, BN2 being connected to each other with a clasp (not shown), the housing 2 is mounted on the mounting target region. It should be noted that it is also possible for the bands BN1, BN2 to be integrally formed with the housing 2.

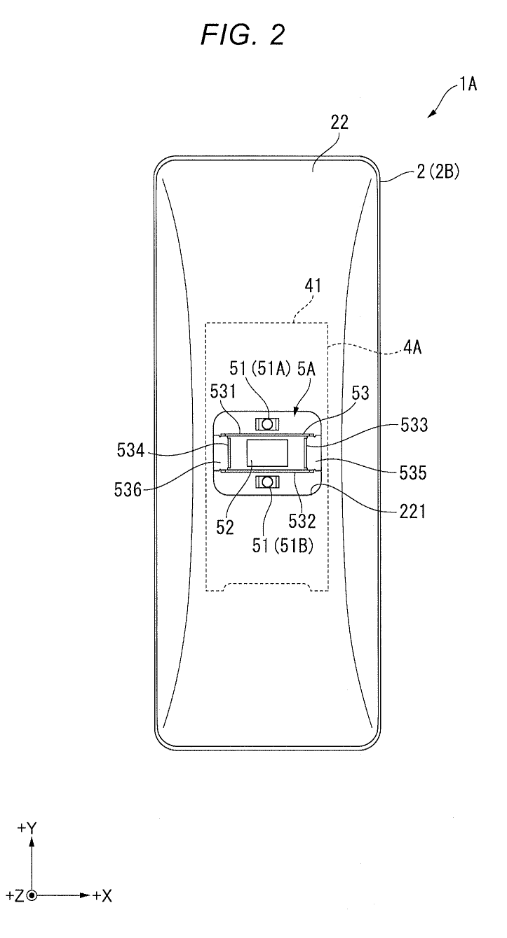

The housing 2 has a front case 2A located on the −Z direction side, and a rear case 2B (see FIG. 2) located on the +Z direction side, and is constituted by these cases combined with each other. The housing 2 has the front side part 21, the rear side part 22 (see FIG. 2) and a lateral side part 23.

The front side part 21 is a region located on the −Z direction side in the housing 2, and is constituted by the front case 2A. The front side part 21 is a region which can be viewed by the user wearing the measurement device 1A. In a roughly central part of the front side part 21, there is disposed a display section DP, and the display section DP is covered with a cover 211.

In the lateral side part 23, a pair of buttons BT constituting an operation section are disposed in a region on the −X direction side.

FIG. 2 is a diagram showing the rear side part 22 of the measurement device 1A.

The rear side part 22 is a region located on the +Z direction side in the housing 2, and is constituted by the rear case 2B. The rear side part 22 is a contact part having contact with the body of the user in the housing 2 when the measurement device 1A is worn by the user.

As shown in FIG. 2, the rear side part 22 is formed to have a warped shape having a central side bulging toward the +Z direction compared to outer edge sides. At the center of the rear side part 22, there is formed an opening part 221 having a roughly rectangular shape for externally exposing a sensor section 5A of a control section 4A housed inside the housing 2. The control section 4A will be described later in detail.

### Internal Configuration of Housing

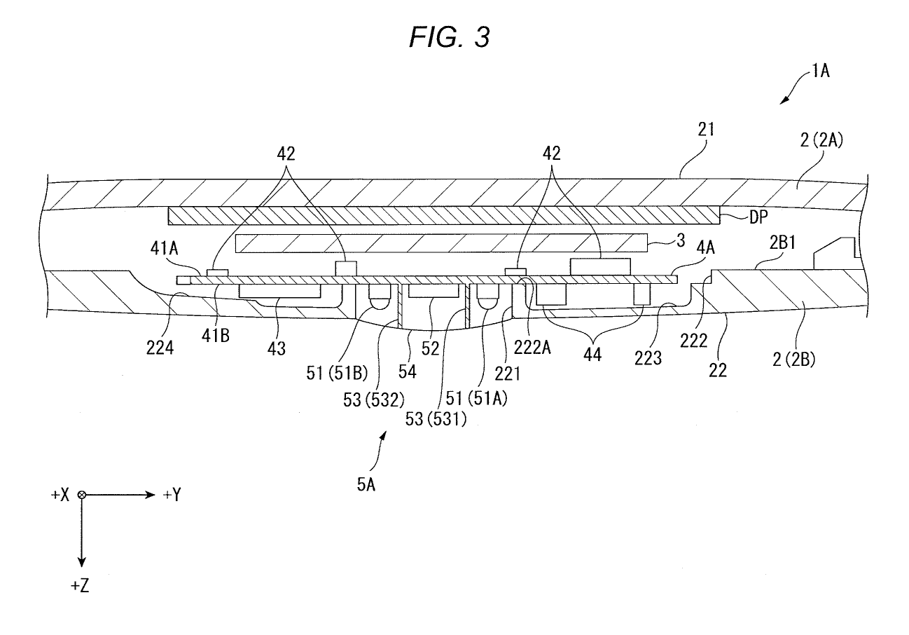

FIG. 3 is a diagram showing an internal configuration of the measurement device 1A, and is in detail a diagram of a cross-section parallel to the Y-Z plane and passing through the center of the measurement device 1A viewed from the −X direction side.

The measurement device 1A has a battery 3 and the control section 4A each housed in the housing 2 as shown in FIG. 3 in addition to the housing 2 and the display section DP.

The battery 3 supplies the electrical power for operating the measurement device 1A. The battery 3 is a secondary cell charged by the electrical power supplied externally under the control by the control section 4A in the present embodiment, but is not limited thereto, and can also be a primary cell.

### Configuration of Control Section

The control section 4A is configured as a control board (a circuit board) for controlling the whole of the measurement device 1A, and is disposed along the X-Y plane in the housing 2. The control section 4A is provided with a board 41 as a rigid board, and an acceleration sensor, a wireless communication circuit, a display control circuit, a storage circuit and a processing circuit each constituted by a plurality of circuit elements 42 disposed on a mounting surface 41A (a first surface) as a surface on the −Y direction side in the board 41. Further, the control section 4A is provided with the sensor section 5A, a signal processor 43 and a plurality of elements 44 each disposed on a mounting surface 41B (a second surface) which is a surface on the +Y direction side in the board 41 and constitutes the two sides of the board 41 with the mounting surface 41A.

The acceleration sensor detects the acceleration acting on the measurement device 1A to output a signal representing a variation of the acceleration thus detected to the processing circuit as a body motion signal representing the body motion of the user. It should be noted that the body motion signal is also used for removal of body motion noise performed by the processing circuit when analyzing the pulse wave signal input from the sensor section 5A to determine the pulse rate.

The wireless communication circuit transmits the biological information and the body motion information based on the detection result of the sensor section 5A and the acceleration sensor to external equipment, and in addition, outputs the information received from the external equipment to the processing circuit under the control by the processing circuit.

The display control circuit display predetermined information on the display section DP under the control by the processing circuit. For example, the display control circuit displays the pulse rate analyzed by the processing circuit on the display section DP.

The storage circuit is formed of a nonvolatile memory such as a flash memory, and stores programs and data necessary for the operation of the measurement device 1A. Besides the above, the storage circuit stores the detection result by the sensor section 5A and the acceleration sensor, and the analysis result by the processing circuit.

The processing circuit is constituted by an arithmetic processing circuit such as a central processing unit (CPU), and functions as a control circuit for controlling the whole of the measurement device 1A automatically or in accordance with an input operation of the user to the operation section such as the buttons BT. For example, the processing circuit determines the pulse rate of the user based on the pulse wave signal input from the sensor section 5A and the body motion signal input from the acceleration sensor as a predetermined process. Further, the processing circuit stores the pulse rate thus determined in the storage circuit, and in addition, displays the pulse rate on the display section DP or transmits the pulse rate to the external equipment with the wireless communication circuit if needed.

### Configuration of Sensor Section

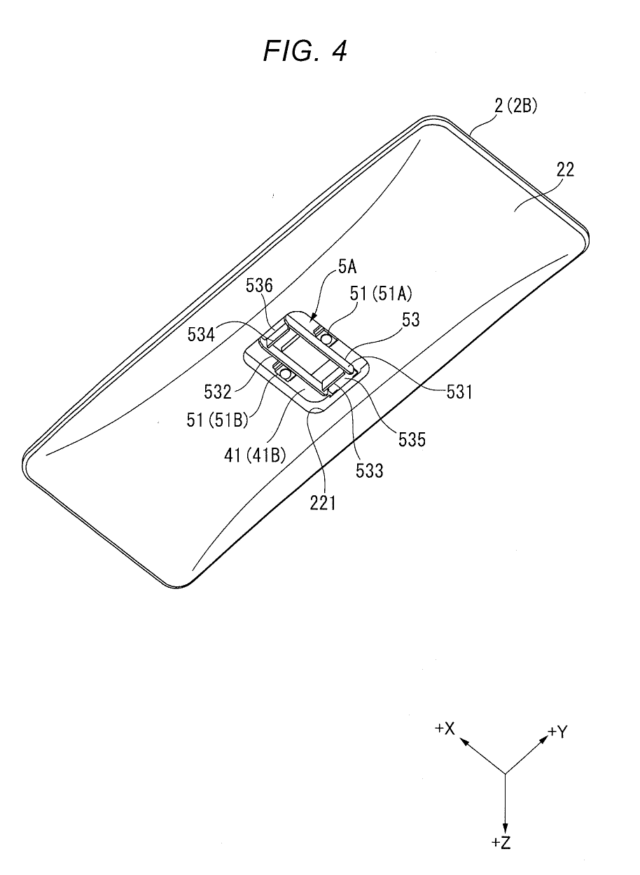

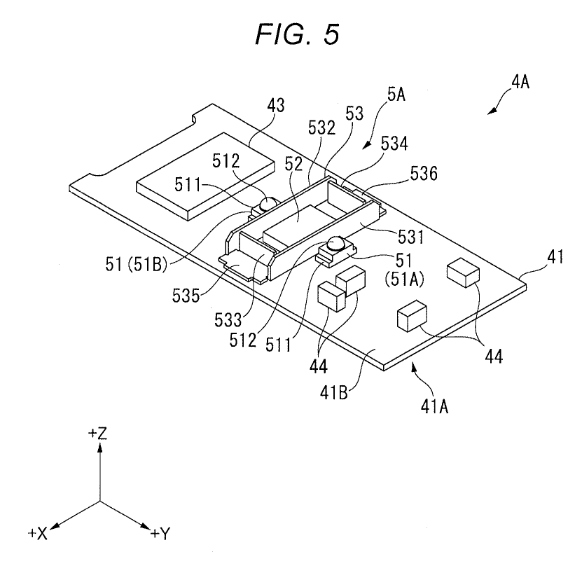

FIG. 4 is a perspective view of the rear case 2B and the sensor section 5A disposed in the control section 4A viewed from the +Z direction side. Further, FIG. 5 is a perspective view of the control section 4A viewed from the +Z direction side. It should be noted that in FIG. 4 and FIG. 5, illustration of a sealing material 54 constituting the sensor section 5A is omitted in order to make the arrangement of the light emitters 51, the light receiver 52 and a light blocking wall 53 in the sensor section 5A easy to understand.

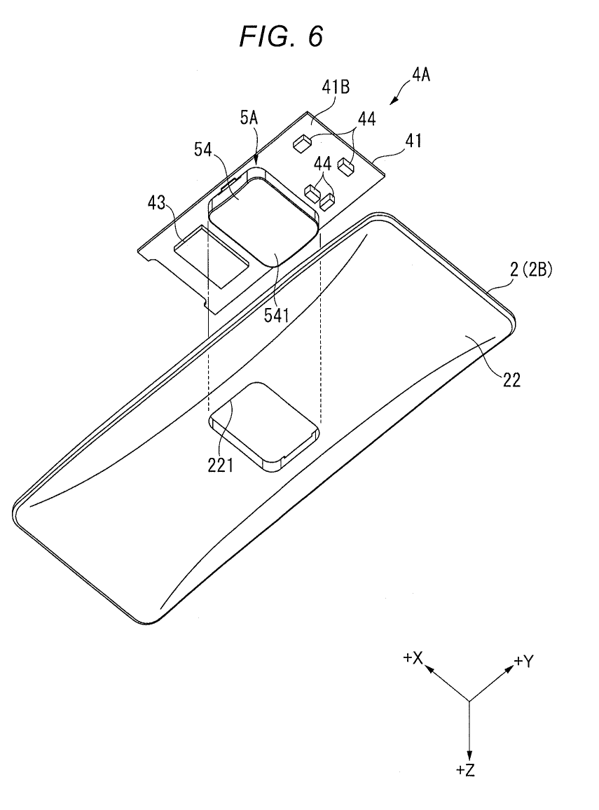

The sensor section 5A irradiates the living body (e.g., the body of the user) with the light, and then outputs a signal representing a variation of the light receiving intensity of the reflected light reflected by the living body as the pulse wave signal representing the pulse wave of the living body. As shown in FIG. 4 and FIG. 5, the sensor section 5A has light emitters 51 (51A, 51B), the light receiver 52, the light blocking wall 53 and the sealing material 54 (see FIG. 6) each disposed on the mounting surface 41B.

### Configuration of Light Emitters

The light emitters 51 emit the light (detection light, e.g., green light) with which the living body is irradiated. There are two light emitters 51 disposed on a straight line passing through the center of the rear side part 22 along the +Y direction across the light receiver 52 from each other with a predetermined distance. In other words, the light emitters 51 consist of the light emitter 51A located on the +Y direction side, and the light emitter 51B located on the −Y direction side. One of the light emitters 51A, 51B corresponds to a first light emitter, and the other thereof corresponds to a second light emitter.

The light emitters 51A, 51B each have a light emitting element (not shown) such as a light emitting diode (LED), a cover section 511 for covering the light emitting element in a surrounding manner, and a lens 512 provided to the cover section 511 so as to cover the light emitting element in the present embodiment. In other words, the light emitters 51A, 51B are each a packaged LED chip.

Although the detailed illustration is omitted, the cover section 511 has a reflecting section surrounding the four sides (the ±X direction sides and the ±Y direction sides) of the light emitting element viewed from a position opposed to the light emitting surface of the light emitting element, and sealing resin with which a space between the light emitting element and the reflecting section is filled. Out of the light emitted from the light emitting element, the light emitted toward the ±X direction sides and the ±Y direction sides is reflected by the reflecting section toward the +Z direction side, and enter the lens 512. It should be noted that the light emitted from the light emitting element toward the +Z direction side also enters the lens 512. The light emitted from the light emitting element and then enters the lens 512 in such a manner is collected by the lens 512 and is then emitted.

It should be noted that the light emitters 51A, 51B can individually be switched between the lighting state and the extinction state under the control by the processing circuit. Therefore, when detecting the pulse wave, at least one of the light emitters 51A, 51B is put on, but the light emitters 51A, 51B are not necessarily required to be put on at the same time.

### Configuration of Light Receiver

The light receiver 52 receives the reflected light which is emitted from the light emitters 51 and is then reflected by the living body, and then outputs the signal representing the variation of the received light intensity of the reflected light as the pulse wave signal representing the waveform of the pulse wave. The light receiver 52 is disposed at the center of a space between the two light emitters 51A, 51B in the +Y direction connecting the two light emitters 51A, 51B to each other. In other words, the dimension between the center of the light emitter 51A and the center of the light receiver 52 is the same as the dimension between the center of the light emitter 51B and the center of the light receiver 52, and the light receiver 52 is disposed at the center of the rear side part 22.

Although the detailed illustration will be omitted, the light receiver 52 has a light receiving element as a photodiode (PD), a cover section for covering the light receiving element and an angle-limiting filter. In other words, the light receiver 52 is a packaged PD chip.

It should be noted that the angle-limiting filter is a filter for transmitting the light the incident angle of which is smaller than a predetermined value wherein the incident angle is an angle of the incident light with respect to the normal line of a filter layer constituting the angle-limiting filter, while preventing the transmission of the light the incident angle of which is equal to or larger than the predetermined value. By disposing such an angle-limiting filter, the disturbance light to be the noise is prevented from entering the light receiver 52. It should be noted that in the present embodiment, the predetermined value described above is set to 30°.

In contrast, the light receiver 52 can also be provided with a wavelength-limiting filter for limiting the wavelength of the light received by the light receiver 52.

### Configuration of Light Blocking Wall

The light blocking wall 53 blocks the light emitted from each of the light emitters 51 and then proceeding toward the light receiver 52 without the intervention of the living body. The light blocking wall 53 is disposed between each of the light emitters 51 (51A, 51B) and the light receiver 52. Specifically, the light blocking wall 53 has a first shield section 531, a second shield section 532, a third shield section 533 and a fourth shield section 534, and is formed to have a rectangular frame shape surrounding the light receiver 52 with the shield sections 531 through 534 viewed from the +Z direction side as a direction perpendicular to the board 41.

The first shield section 531 is located on the +Y direction side with respect to the light receiver 52, and is located between the light emitter 51A and the light receiver 52. In other words, the first shield section 531 is located at a position on the light emitter 51A side with respect to the light receiver 52, and on the opposite side to the light emitter 51B side.

The second shield section 532 is located on the −Y direction side with respect to the light receiver 52, and is located between the light emitter 51B and the light receiver 52. In other words, the second shield section 532 is located at a position on the light emitter 51B side with respect to the light receiver 52, and on the opposite side to the light emitter 51A side.

The third shield section 533 is located on the +X direction side with respect to the light receiver 52, and the fourth shield section 534 is located on the −X direction side with respect to the light receiver 52.

Further, the light blocking wall 53 has a bending part 535 to be connected to the third shield section 533, and a bending part 536 to be connected to the fourth shield section 534. These bending parts 535, 536 are each an attachment section shaped like a flat plate along the X-Y plane, and by surfaces on the −Z direction side of the bending parts 535, 536 being attached to the mounting surface 41B with solder or the like, the light blocking wall 53 is attached to the mounting surface 41B.

### Configuration of Sealing Material

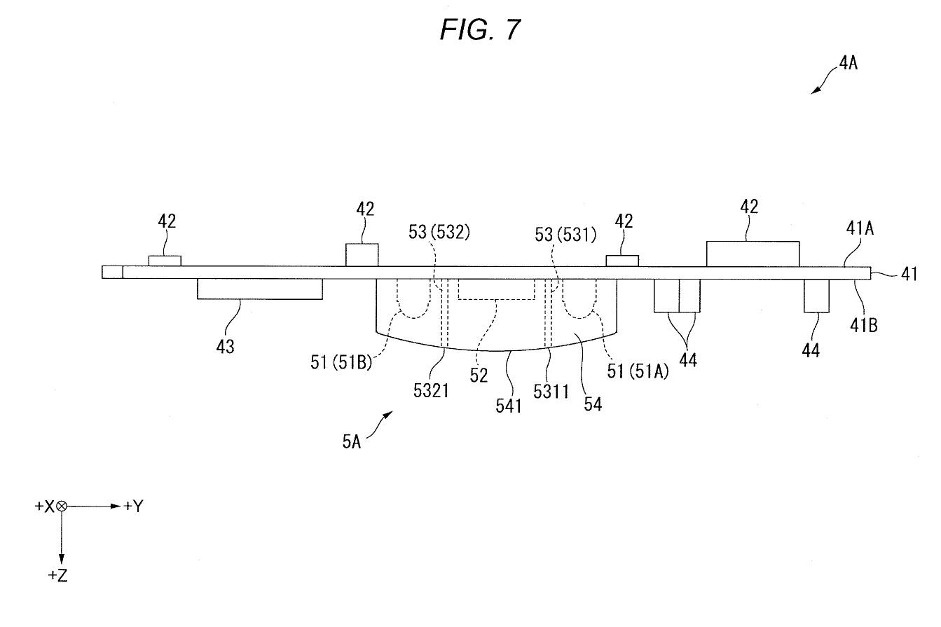

FIG. 6 is a perspective view showing an arrangement of the control section 4A to the rear case 2B, and is an exploded perspective view showing the rear case 2B and the control section 4A in other words. Further, FIG. 7 is a diagram of the control section 4A viewed from the lateral side (the −X direction side).

As shown in FIG. 6 and FIG. 7, the sealing material 54 seals the light emitters 51, the light receiver 52 and the light blocking wall 53 on the mounting surface 41B of the board 41 to thereby protect the light emitters 51, the light receiver 52 and the light blocking wall 53. The sealing material 54 is exposed outside the housing 2 via the opening part 221 provided to the rear case 2B when the control section 4A is attached to the rear case 2B.

Such a sealing material 54 is formed of the sealing resin having a light transmissive property for transmitting the light emitted from the light emitters 51 and the light entering the light receiver 52. Therefore, the light emitted from the light emitters 51 is emitted outside the housing 2 via the sealing material 54, and further, the light to be received by the light receiver 52 enters the light receiver 52 via the sealing material 54.

It should be noted that sealing does not necessarily means that the whole of the sealing target as the target of sealing is enclosed inside the sealing material 54. For example, providing the sealing material 54 encloses the light emitters 51 and the light receiver 52 as the sealing target inside the sealing material 54, it is also possible for a part of the light blocking wall 53 similarly as the sealing target to slightly project outside the sealing material 54.

Such a sealing material 54 has a contact surface 541 which is a surface on the +Z direction side, and can have contact with the living body when the measurement device 1A is mounted on the living body. The contact surface 541 is also an emission surface from which the light having been emitted from the light emitters 51 is emitted mainly outside the sealing material 54, and further, is also a plane of incidence through which the light entering the light receiver 52 mainly enters the sealing material 54 from the outside (the living body). In other words, the contact surface 541 is an incident/emission surface with respect to the sealing material 54.

As shown in FIG. 7, such a contact surface 541 is formed to have a convexly curved shape in which the central part in the +X direction and the +Y direction bulges in the +Z direction from the outer edge sides. The shape of the contact surface 541 is a shape calculated to collect the light emitted from the light emitters 51 to irradiate the living body with the collected light.

Here, the position of the tip part in the +Z direction as the projecting direction from the mounting surface 41B in the light blocking wall 53 is not only located on the +Z direction side from the end part on the +Z direction side in the light emitters 51 (51A, 51B), but also located at roughly the same position as that of the contact surface 541. For example, as shown in FIG. 7, the position of the tip part 5311 in the +Z direction in the first shield section 531 is not only located on the +Z direction side from the end part on the +Z direction side in the light emitter 51A, but also located at roughly the same position as that of the contact surface 541. Further, the position of the tip part 5321 in the +Z direction in the second shield section 532 is not only located on the +Z direction side from the end part on the +Z direction side in the light emitter 51B, but also located at roughly the same position as that of the contact surface 541. It should be noted that although not shown in the drawings, the positions of the tip parts on the +Z direction side in the third shield section 533 and the fourth shield section 534 are also roughly the same position as that of the contact surface 541.

This is because if the positions of the tip parts (e.g., the tip parts 5311, 5321) of the light blocking wall 53 are located on the −Z direction side from the contact surface 541, there is a possibility that the light emitted from the light emitters 51A, 51B is internally reflected by the contact surface 541 as the boundary surface and enters the light receiver 52 beyond the light blocking wall 53. The positions of the tip parts of the light blocking wall 53 are set at roughly the same position as that of the contact surface 541 so that the light internally reflected in such a manner is also blocked. It should be noted that even in the case in which the tip part of the light blocking wall 53 projects outside the sealing material 54 (the contact surface 541), it is defined that the light blocking wall 53 is sealed by the sealing material 54 as described above.

In order to make the position of the contact surface 541 and the position of the tip part of the light blocking wall 53 coincide with each other as described above, it is possible to adopt a method of forming the sealing material 54 using a mold or the like so that the positions roughly coincide with each other, or a method of grinding down the sealing material 54 thus formed so that the positions roughly coincide with each other.

### Configuration of Signal Processor

As shown in FIGS. 5 through 7, the signal processor 43 is disposed in a region on the −Y direction side with respect to the sensor section 5A in the mounting surface 41B. The signal processor 43 is a circuit element for processing the pulse wave signal, and is specifically an analog front end (AFE) for processing the pulse wave signal.

Such a signal processor 43 performs processes such as amplification, noise removal and A/D conversion on the pulse wave signal input from the light receiver 52. In other words, the signal processor 43 has a primary amplifying section, a filter section, a secondary amplifying section, an A/D conversion section and a communication section. Then, the signal processor 43 outputs the pulse wave signal thus processed to the processing circuit described above.

It should be noted that the plurality of elements 44 disposed in the region on the +Y direction side with respect to the sensor section 5A in the mounting surface 41B corresponds to circuit elements such as resistors, transistors and capacitors. The arrangement positions of the elements 44 and the arrangement position of the signal processor 43 described above can be reversed from each other. Specifically, it is also possible to dispose the signal processor 43 on the +Y direction side with respect to the sensor section 5A in the mounting surface 41B, and dispose the plurality of elements 44 on the −Y direction side with respect to the sensor section 5A.

### Configuration of Rear Case

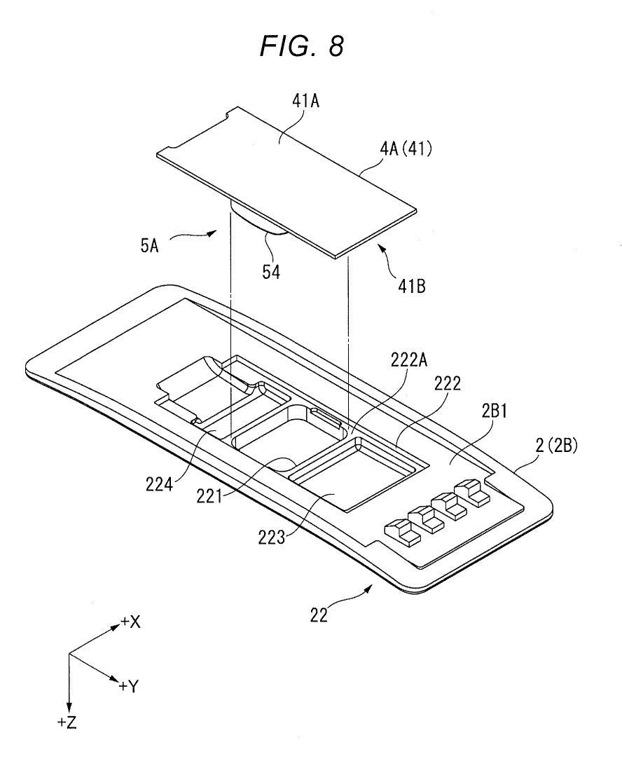

FIG. 8 is a perspective view showing a configuration of the rear case 2B in which the control section 4A is disposed. In other words, FIG. 8 is a perspective view of the rear case 2B viewed from the opposite side (the −Z direction side) to the light emission side. It should be noted that in FIG. 8, illustration of the circuit elements 42 mounted on the mounting surface 41A is omitted.

The rear case 2B constitutes the rear side part 22 of the housing 2 for housing the battery 3 and the control section 4A as described above. As shown in FIG. 8, the rear case 2B has a placement part 222 in a roughly central part of a surface 2B1 (the surface 2B1 as an inside surface of the rear side part 22) on the −Z direction side, wherein the control section 4A is disposed in the placement part 222 from the −Z direction side with the mounting surface 41B facing to the +Z direction side.

The placement part 222 is formed to have a rectangular shape corresponding to the board 41 of the control section 4A viewed from the −Z direction side, and have a recessed shape recessed toward the +Z direction side. In the placement part 222, a bottom part 222A as a region on the +Z direction side has contact with the mounting surface 41B of the substrate 41 to support the control section 4A.

At the center of the bottom part 222A, there is formed the opening part 221 described above for exposing the sensor section 5A outside the housing 2, and on the ±Y direction sides of the opening part 221, there are formed recessed parts 223, 224 further recessed toward the +Z direction.

The recessed part 223 located on the +Y direction side is a recessed part where at least some of the plurality of elements 44 disposed on the mounting surface 41B are disposed inside. The recessed part 224 located on the −Y direction side is a recessed part where at least a part of the signal processor 43 disposed on the mounting surface 41B is disposed inside.

### Advantages of First Embodiment

According to the biological information measurement device 1A related to the present embodiment described hereinabove, the following advantages can be exerted.

The biological information measurement device 1A is provided with the board 41, the circuit elements 42 disposed on the mounting surface 41A as the first surface of the board 41 to execute a predetermined process, and the sensor section 5A disposed on the mounting surface 41B forming the two sides of the board 41 with the mounting surface 41A to detect the biological information. The sensor section 5A has the light emitters 51 for irradiating the living body with the light, the light receiver 52 for receiving the reflected light reflected by the living body, and the sealing material 54 which has the light transmissive property, contact with the living body, and seals the light emitters 51 and the light receiver 52 to the board 41.

According to this configuration, it is possible to protect the light emitters 51 and the light receiver 52 with the sealing material 54.

Further, since the sealing material 54 has contact with the living body, it is possible to shorten the distance from the light emitters 51 to the living body and the distance from the living body to the light receiver 52 compared to the case in which the light transmissive member covering the sensor section 5A is provided separately from the sensor section 5A. Therefore, it is possible to suppress the attenuation of the light with which the living body is irradiated by the light emitters 51, and the attenuation of the light entering the light receiver 52 from the living body, and thus, it is possible to suppress the reduction of the received light intensity in the light receiver 52. Therefore, it is possible to improve the detection accuracy of the biological information by the measurement device 1A.

Besides the above, since the light transmissive member covering the sensor section 5A does not exist, it is possible to achieve reduction in thickness of the measurement device 1A.

The circuit elements 42 disposed on the mounting surface 41A include the processing circuit for determining the biological information based on the signal output from the light receiver 52. According to this configuration, since the light emitters 51 and the light receiver 52 are disposed on the mounting surface 41B of the board 41, and the circuit elements 42 including the processing circuit described above are disposed on the mounting surface 41A on the opposite side to the mounting surface 41B, the function (i.e., the function of detecting and measuring the biological information) as the biological information measurement device can be realized with the configuration provided to the board 41. Therefore, since there is no need for separately providing the board provided with the light emitters 51 and the light receiver 52 and the board provided with the processing circuit described above, the configuration of the measurement device 1A can be simplified, and in addition, it is possible to achieve further reduction in size of the measurement device 1A.

The sensor section 5A has the light blocking wall 53 disposed between the light emitters 51 and the light receiver 52 in addition to the light emitters 51 and the light receiver 52. The light blocking wall 53 is sealed by the sealing material 54 together with the light emitters 51 and the light receiver 52. According to this configuration, the light directly proceeding from each of the light emitters 51 toward the light receiver 52 without the intervention of the living body can be blocked by the light blocking wall 53. Therefore, it is possible to improve the detection accuracy and the measurement accuracy of the biological information.

Further, since the light blocking wall 53 is sealed by the sealing material 54 together with the light emitters 51 and the light receiver 52, there is no need for separately disposing the light blocking wall 53 outside the sealing material 54. Therefore, it is possible to achieve the reduction in thickness of the biological information measurement device 1A in combination with the fact that it is possible to shorten the distance between the contact surface 541 and the light emitters 51 and the distance between the contact surface 541 and the light receiver 52.

The light emitters 51 consist of the light emitters 51A, 51B as the first light emitter and the second light emitter. The light receiver 52 is disposed between the light emitters 51A, 51B, and the light blocking wall 53 is disposed between the light emitter 51A and the light receiver 52 and between the light emitter 51B and the light receiver 52. According to this configuration, by using the light emitter at the position suitable for the detection of the biological information out of the light emitters 51A, 51B, it is possible to improve the detection accuracy of the biological information. In contrast, since it is possible to increase the received light intensity in the light receiver 52 by using each of the light emitters 51A, 51B, even in this case, it is possible to improve the detection accuracy of the biological information.

Further, due to the light blocking wall 53, it is possible to block the light directly proceeding from the light emitter 51A toward the light receiver 52 and the light directly proceeding from the light emitter 51B toward the light receiver 52. Therefore, since it is possible to prevent the light having not passed through the living body from entering the light receiver 52, it is possible to further improve the detection accuracy of the biological information.

Here, since the light emitters 51 and the light receiver 52 are sealed by the sealing material 54, there is a possibility that a part of the light emitted from each of the light emitters 51 proceeds inside the sealing material 54 while repeating the internal reflection, and then enters the light receiver 52 from a different side from the light emitter 51 side in the light receiver 52.

To cope with the above, the light blocking wall 53 surrounds the light receiver 52 viewed from the +Z direction side as the direction perpendicular to the substrate 41. According to this configuration, it is possible not only to block the light emitted from each of the light emitters 51 and then proceeding inside the sealing material 54 without the intervention of the living body toward the light receiver 52 with the light blocking wall 53, but also to block the disturbance light entering the sealing material 54 and then proceeding toward the light receiver 52 with the light blocking wall 53. Therefore, it is possible to further improve the detection accuracy of the biological information.

Here, in the case in which the light emitters 51 emit the light with a predetermined emission angle, there is a possibility that a part of the light emitted from each of the light emitters 51 is internally reflected by the contact surface 541. In such a case, if the tip part on the projecting direction side from the board 41 is located on the board 41 side from the contact surface 541 in the light blocking wall 53, there arises the possibility that the part of the light enters the light receiver 52 beyond the light blocking wall 53.

To cope with the above, the positions of the tip parts (e.g., the tip parts 5311, 5321) on the projecting direction side from the board 41 in the light blocking wall 53 roughly coincide with the position of the contact surface 541 as the surface from which the light emitted from the light emitters 51 is emitted to the outside in the sealing material 54. According to this configuration, it is possible to block the light internally reflected by the contact surface 541 with the light blocking wall 53, and thus, it is possible to prevent the light internally reflected from entering the light receiver 52. Therefore, the deterioration of the detection accuracy of the biological information can be suppressed.

The contact surface 541 as the surface from which the light emitted from the light emitters 51 is emitted to the outside in the sealing material 54 is a curved surface. In the detailed description, the contact surface 541 is a convexly curved surface in which the central area projects toward the +Z direction from the outer edge. According to this configuration, it is possible to make the sealing material 54 function as a convex lens. Therefore, it is possible to collect the light emitted from the light emitters 51 to irradiate the living body with the collected light. Therefore, it is possible to further improve the detection accuracy of the biological information.

The biological information measurement device 1A is provided with the signal processor 43 disposed on the mounting surface 41B to process the signal output from the light receiver 52, and the housing 2 for housing the board 41. The rear case 2B constituting the housing 2 has the recessed part 224 where at least a part of the signal processor 43 is disposed inside. According to this configuration, it is possible to reduce the thickness of the rear case 2B, and by extension, the thickness of the housing 2 compared to the case where the recessed part 224 is not provided. Therefore, it is possible to achieve further reduction in thickness of the biological information measurement device 1A.

### Second Embodiment

Then, a second embodiment of the invention will be described.

A biological information measurement device according to the present embodiment has substantially the same configuration as that of the biological information measurement device 1A described in the first embodiment, but is different from the biological information measurement device 1A in the point that the configuration of the light emitter and the light receiver is different. It should be noted that in the description below, a part which is the same or substantially the same as the part having already been described is denoted by the same reference symbol, and the description thereof will be omitted.

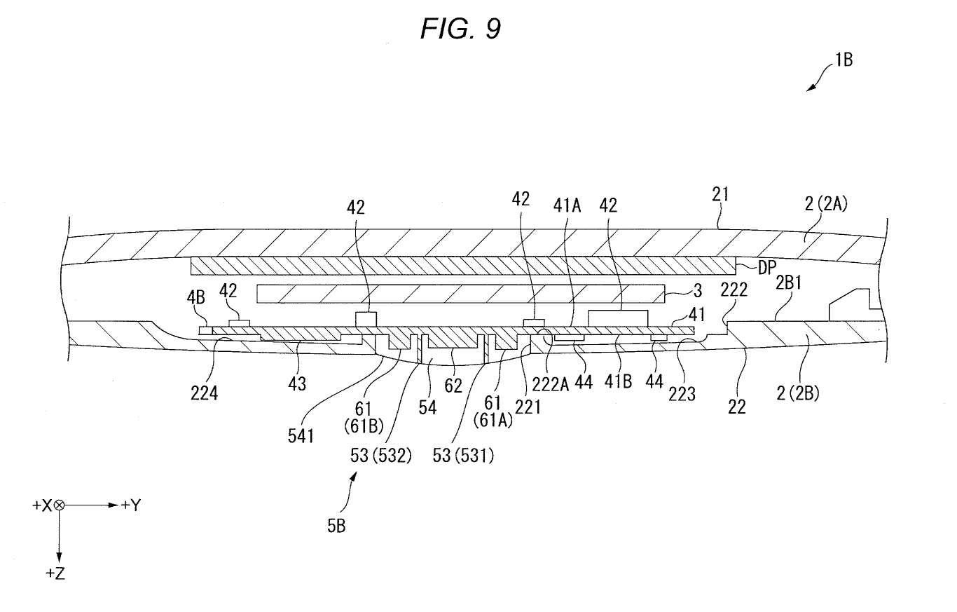

FIG. 9 is a diagram showing an internal configuration of the biological information measurement device 1B according to the present embodiment, and is in detail a diagram of a cross-section parallel to the Y-Z plane and passing through the center of the biological information measurement device 1B viewed from the −X direction side.

As shown in FIG. 9, the biological information measurement device 1B has substantially the same configuration and functions as those of the biological information measurement device 1A except the fact that a control section 4B is provided instead of the control section 4A, and the control section 4B has substantially the same configuration and functions as those of the control section 4A except the fact that a sensor section 5B is provided instead of the sensor section 5A.

The sensor section 5B has substantially the same configuration and functions as the sensor section 5A described above except the fact that light emitters 61 and a light receiver 62 are provided instead of the light emitters 51 and the light receiver 52.

Similarly to the light emitters 51, the light emitters 61 emit the light (detection light, e.g., green light) with which the living body is irradiated. The light emitters 61 are respectively disposed at the same positions as those of the light emitters 51 in the mounting surface 41B. Specifically, the two light emitters 61 are disposed with a predetermined distance in the +Y direction. In other words, the light emitters 61 consist of a light emitter 61A and a light emitter 61B.

The light emitter 61A is located on the +Y direction side with respect to the light receiver 62 located at the center in the sensor section 5B. Further, the light emitter 61B is located on the −Y direction side with respect to the light receiver 62.

These light emitters 61A, 61B correspond respectively to the first light emitter and the second light emitter according to the invention, and are each formed of a light emitting element such as a light emitting diode (LED) in the present embodiment. In other words, the light emitters 61A, 61B are each an LED bare chip not packaged, and are attached to the mounting surface 41B with bare chip mounting. It should be noted that as the bare chip mounting, there can be cited wire bonding, tape automated bonding (TAB) using a film having lead wires, and flip-chip bonding using bumps.

Similarly to the light receiver 52 described above, the light receiver 62 receives the reflected light reflected by the living body, and then outputs the signal corresponding to the received light intensity of the reflected light as the pulse wave signal representing the waveform of the pulse wave. The light receiver 62 is disposed at the same position as that of the light receiver 52 in the mounting surface 41B. Specifically, the light receiver 62 is disposed between the light emitters 61A, 61B, the dimension between the center of the light emitter 61A and the center of the light receiver 62 is the same as the dimension between the center of the light emitter 61B and the center of the light receiver 62, and the light receiver 62 is disposed at the center of the rear side part 22.

Such a light receiver 62 is formed of a light receiving element such as a PD in the present embodiment. In other words, the light receiver 62 is a PD bare chip not packaged, and is attached on the mounting surface 41B with the bare chip mounting.

Also in such a sensor section 5B, the sealing material 54 seals the light emitters 61 (61A, 61B), the light receiver 62 and the light blocking wall 53 on the mounting surface 41B to protect these sections.

Further, although the detailed illustration is omitted in FIG. 9, also in the sensor section 5B, the positions of the tip parts on the +Z direction side of the light blocking wall 53 roughly coincide with the position of the contact surface 541 formed to have a convexly curved surface shape in the sealing material 54.

### Advantages of Second Embodiment

According to the measurement device 1B according to the present embodiment described hereinabove, in addition to the advantages substantially the same as those of the measurement device 1A described above, the following advantages can be obtained.

In the sensor section 5B, the light emitters 61 and the light receiver 62 sealed by the sealing material 54 together with the light blocking wall 53 are each formed of the bare chip not packaged. According to this configuration, since the bare chip is smaller in thickness dimension (the rising dimension from the board 41 in the case of disposing the chip on the board 41) than that of the packaged chip, it is possible to make the thickness dimensions of the light emitters 61 and the light receiver 62 smaller compared to the light emitters 51 and the light receiver 52, and thus, it is possible to make the thickness dimension of the sealing material 54 smaller. Therefore, it is possible to further reduce the thickness of the sensor section 5B, and by extension, the thickness of the biological information measurement device 1B.

It should be noted that the light emitters 61 each formed of the bare chip emit the light mainly from the surface facing to the +Z direction side, and also emit the light from the surfaces on the ±X direction sides and the ±Y direction sides. Meanwhile, the light receiver 62 formed of the bare chip receives the light mainly in the surface facing to the +Z direction side, and also receives the light in the surfaces on the ±X direction sides and the ±Y direction sides. Therefore, there is a possibility that the light emitted from the surfaces on the ±X direction sides and the ±Y direction sides in the light emitters 61 proceeds inside the sealing material 54 due to the internal reflection or the like, and is then received in the surfaces on the ±X direction sides and the ±Y direction sides in the light receiver 62.

To cope with the above, since the light blocking wall 53 is formed to have a frame shape surrounding the light receiver 62 on the ±X direction sides and the ±Y direction sides, the light emitted from the surfaces on the ±X direction sides and the ±Y direction sides in the light emitters 61 and then proceeding inside the sealing material 54 toward the light receiver 62 can be blocked by the light blocking wall 53. Therefore, the advantages obtained from the fact that the light blocking wall 53 is formed to have the frame shape surrounding the light receiver 62 can be exerted in good condition.

### Third Embodiment

Then, a third embodiment of the invention will be described.

A biological information measurement device according to the present embodiment has substantially the same configuration as that of the biological information measurement device 1B described in the second embodiment, but is different from the biological information measurement device 1B in the point that the shape of the sealing material constituting the biological information detection sensor is different. It should be noted that in the description below, a part which is the same or substantially the same as the part having already been described is denoted by the same reference symbol, and the description thereof will be omitted.

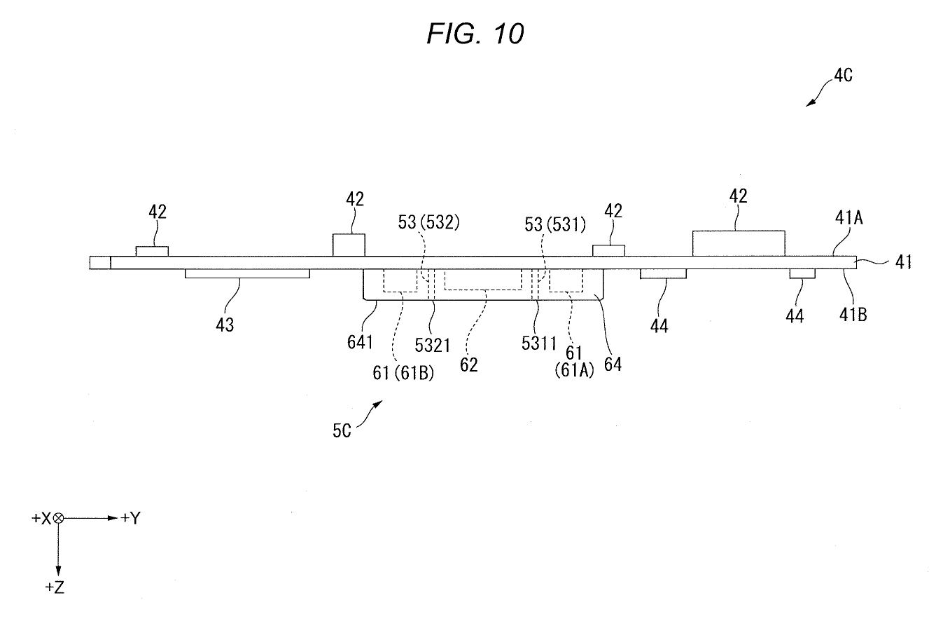

FIG. 10 is a side view showing a control section 4C provided to the biological information measurement device according to the present embodiment. In the detailed description, FIG. 10 is a side view of the control section 4C viewed from the −X direction side.

The biological information measurement device according to the present embodiment has substantially the same configuration and functions as those of the biological information measurement device 1B except the fact that a control section 4C is provided instead of the control section 4B, and the control section 4C has substantially the same configuration and functions as those of the control section 4B except the fact that a sensor section 5C is provided instead of the sensor section 5B.

The sensor section 5C has substantially the same configuration and functions as those of the sensor section 5B except the fact that a sealing material 64 is provided instead of the sealing material 54. Specifically, the sensor section 5C has the light emitters 61 (61A, 61B), the light receiver 62 and the light blocking wall 53 each disposed on the mounting surface 41B of the board 41, and the sealing material 64 for sealing the light emitters 61, the light receiver 62 and the light blocking wall 53 on the mounting surface 41B.

The sealing material 64 is formed of the sealing resin having a light transmissive property similar to that of the sealing material 54. The surface on the +Z direction side in the sealing material 64 is the incident/emission surface from which the light emitted from the light emitters 61 is emitted, and which the light passing through the living body enters, and is a contact surface 641 capable of having contact with the living body (e.g., the body of the user). The contact surface 641 is a plane (a plane roughly perpendicular to the +Z direction) roughly parallel to the X-Y plane.

It should be noted that also in the sensor section 5C, the positions of the tip parts (e.g., the tip parts 5311, 5321) on the +Z direction side as the tip parts in the projecting direction from the board 41 in the light blocking wall 53 roughly coincide with the position of the contact surface 641.

### Advantages of Third Embodiment

According to the biological information measurement device according to the present embodiment described hereinabove, in addition to the advantages substantially the same as those of the biological information measurement device 1B, the following advantages can be obtained.

The contact surface 641 in the sealing material 64 is a flat surface. According to this configuration, it is possible to make the thickness dimension in the sensor section 5C smaller. Therefore, it is possible to achieve further reduction in thickness of the sensor section 5C, and by extension, it is possible to achieve further reduction in thickness of the biological information measurement device.

It should be noted that the sensor section 5C is assumed to have the light emitters 61 and the light receiver 62 in the example shown in FIG. 10, but can also be provided with the configuration of having the light emitters 51 and the light receiver 52.

### Modifications of Embodiment

The invention is not limited to each of the embodiments described above, but includes modifications, improvements, and so on within the range in which the advantages of the invention can be achieved.

In the first and second embodiments described above, it is assumed that the contact surface 541 of the sealing material 54 is the convexly curved surface, and in the third embodiment, it is assumed that the contact surface 641 of the sealing material 64 is a flat surface. However, this is not a limitation, and the shape of the surface on the emission direction side of the light in the sealing material can also be another shape.

For example, the contact surfaces 541, 641 can also be a concavely curved surface. In the case in which the contact surfaces 541, 641 are concavely curved surfaces, it is possible to make it easy to diffuse the light emitted from the light emitters 51, 61 to the outside of the sealing materials 54, 64, and it is possible to make it easy to converge the light entering the sealing materials 54, 64 on the light receivers 52, 63.

By changing the shape and the curvature of the contact surface 541, 641 as described above, it is possible to effectively irradiate the living body with the light emitted from the light emitters 51, 61 in accordance with the purpose of the biological information measurement device, and in addition, it is possible to make the light entering the contact surface 541, 641 from the living body efficiently enter the light receiver 52, 62.

In each of the embodiments described above, it is assumed that the signal processor 43 and the plurality of elements 44 are disposed on the mounting surface 41B on which the sensor section 5A through 5C is located in the board 41. However, this is not a limitation, among the plurality of constituents disposed on the mounting surface 41B, at least one of the constituents except the sensor section 5A through 5C can be disposed on the mounting surface 41A on the opposite side to the mounting surface 41B.

In the first embodiment described above, it is assumed that the sensor section 5A is provided with the two light emitters 51A, 51B and the one light receiver 52, and in the second and third embodiments described above, it is assumed that the sensor section 5B, 5C is provided with the two light emitters 61A, 61B and the one light receiver 62. However, this is not a limitation, and the number of the light emitters and the number of the light receivers can arbitrarily be changed. For example, one light emitter can be provided with respect to one light receiver, or three or more light emitters can be provided with respect to one light receiver. Further, two or more light receivers can be provided with respect to one light emitter.

Further, it is also possible to provide a plurality of sets of light emitter and the light receiver, each of the sets including at least one light emitter and at least one light receiver. In this case, the sets can be sealed with a plurality of sealing materials set by set.

Further, the layout of the light emitters and the light receivers can arbitrarily be changed, and for example, a plurality of light emitters can be arranged at intervals in the +X direction.

In the second and third embodiments described above, it is assumed that the sensor sections 5B, 5C each have the light emitters 61 as the bare chips and the light receiver 62 as the bare chip. However, this is not a limitation, and it is also possible to use the bare chip as either one of the light emitter and the light receiver. For example, it is also possible for the sensor section to have a configuration having a combination of the light emitters 61 as the bare chips and the light receiver 52 packaged, or to have a configuration having a combination of the light emitters 51 packaged and the light receiver 62 as the bare chip. Further, in the case in which the sensor section has a plurality of light emitters, it is possible to adopt the configuration in which all of the light emitters are formed of the bare chips, or it is also possible to adopt the configuration in which some of the light emitters are formed of the bare chips and the rest of the light emitters are formed of the packaged chips. Similarly, in the case in which the sensor section has a plurality of light receivers, it is possible to adopt the configuration in which all of the light receivers are formed of the bare chips, or it is also possible to adopt the configuration in which some of the light receivers are formed of the bare chips and the rest of the light receivers are formed of the packaged chips.

Further, the board 41 is assumed to be the rigid board, but can also be a flexible printed circuit (FPC) board.

In each of the embodiments described above, it is assumed that the light blocking wall 53 is formed to have the rectangular frame shape surrounding the light receiver 52, 62. However, this is not a limitation, and the shape of the light blocking wall 53 can be a ring-like shape, or can also be a polygonal shape other than the rectangular shape.

Further, the light blocking wall 53 is not required to have the shape surrounding the light receiver 52, 62. Specifically, providing the light blocking wall is located between the light emitters and the light receiver, and the light emitted from the light emitter and then directly entering the light receiver can be blocked, the shape of the light blocking wall can arbitrarily be changed. For example, in the sensor section having one light emitter and one light receiver, if the light blocking wall exists between the light emitter and the light receiver, it is not required to dispose the light blocking wall (the shield section) in a different direction from the direction from the light receiver toward the light emitter, and further, it is also possible to the light blocking wall to be formed to have a frame shape independently of the number of the light emitters and the number of the light receivers. Further, a gap can be provided to a part of the light blocking wall, or the light blocking wall can also be divided into a plurality of shield sections (light blocking walls).

In addition, the light blocking wall 53 is not required to be sealed by the sealing material 54, 64, and the light blocking wall can be eliminated from the sensor section.

In each of the embodiments described above, it is assumed that the positions of the tip parts on the +Z direction side (the projecting direction side from the board 41) in the light blocking wall 53 coincide with the position of the contact surface 541, 641. However, this is not a limitation, and the positions of the tip parts in the light blocking wall 53 can arbitrarily be changed. It should be noted that as described above, it is preferable for the tip parts of the light blocking wall to be located on the +Z direction side (the light emission side) from the light emitter.

In each of the embodiments described above, it is assumed that the control section 4A through 4C is provided with the acceleration sensor for detecting the acceleration acting on the measurement device. However, this is not a limitation, and it is not required to provide the acceleration sensor to the control section 4A through 4C, and further, even in the case in which the acceleration sensor is provided, it is also possible to provide the acceleration sensor to other constituents (e.g., the bands BN1, BN2) than the board 41. Further, it is also possible for the biological information measurement device to be equipped with other sensors such as a position sensor (e.g., a GPS sensor) capable of measuring the positional information.

Further, it is assumed that the control section 4A through 4C has the acceleration sensor, the wireless communication circuit, the display control circuit, the storage circuit and the processing circuit each constituted by the circuit elements 42. However, this is not a limitation, and at least one of the acceleration sensor, the wireless communication circuit, the display control circuit, the storage circuit and the processing circuit can be eliminated, and further, it is also possible to provide a circuit element to the board 41 for executing another process instead of at least one of these constituents, or in addition to these constituents.

In each of the embodiments described above, it is assumed that the control section 4A through 4C of the biological information measurement device 1A, 1B detects the pulse wave as one type of the biological information, and then determines the pulse rate as another type of the biological information based on the pulse wave signal representing the pulse wave thus detected. In other words, it is assumed that the biological information measurement device described above measures the pulse wave and the pulse rate as the biological information. However, this is not a limitation, and the biological information the biological information measurement device according to the invention can detect and measure is not limited to the pulse wave and the pulse rate. It is also possible to apply the invention to a biological information measurement device for measuring other types of biological information such as heart rate variability (HRV), a pulse interval (R-R interval (RRI)), blood pressure, a blood sugar level, an amount of activity, calorie consumption, or maximum oxygen uptake (VO₂max).

What is claimed is:

1. A biological information measurement device adapted to measure biological information of a living body, comprising:
a board having a first surface and a second surface adapted to form two surfaces of the board with the first surface;
a circuit element disposed on the first surface, and adapted to execute a predetermined process; and
a sensor having
a light emitter adapted to irradiate the living body with light,
a light receiver adapted to receive reflected light reflected by the living body, and
a sealing material having a light transmissive property, having contact with the living body, and adapted to seal the light emitter and the light receiver on the board, the light emitter, the light receiver and the sealing material being disposed on the second surface.

2. The biological information measurement device according to claim 1, wherein
the circuit element includes a processing circuit adapted to determine the biological information based on a signal output from the light receiver.

3. The biological information measurement device according to claim 1, wherein
at least either one of the light emitter and the light receiver is constituted by a bare chip.

4. The biological information measurement device according to claim 2, wherein
at least either one of the light emitter and the light receiver is constituted by a bare chip.

5. The biological information measurement device according to claim 1, wherein
the sensor has a light blocking wall disposed between the light emitter and the light receiver, and
the light blocking wall is sealed by the sealing material together with the light emitter and the light receiver.

6. The biological information measurement device according to claim 2, wherein
the sensor has a light blocking wall disposed between the light emitter and the light receiver, and
the light blocking wall is sealed by the sealing material together with the light emitter and the light receiver.

7. The biological information measurement device according to claim 3, wherein
the sensor has a light blocking wall disposed between the light emitter and the light receiver, and
the light blocking wall is sealed by the sealing material together with the light emitter and the light receiver.

8. The biological information measurement device according to claim 4, wherein
the sensor section has a light blocking wall disposed between the light emitter and the light receiver, and
the light blocking wall is sealed by the sealing material together with the light emitter and the light receiver.

9. The biological information measurement device according to claim 5, wherein
the light emitter has a first light emitter and a second light emitter,
the light receiver is disposed between the first light emitter and the second light emitter, and
the light blocking wall is disposed between the first light emitter and the light receiver and between the second light emitter and the light receiver.

10. The biological information measurement device according to claim 6, wherein
the light emitter has a first light emitter and a second light emitter,
the light receiver is disposed between the first light emitter and the second light emitter, and
the light blocking wall is disposed between the first light emitter and the light receiver and between the second light emitter and the light receiver.

11. The biological information measurement device according to claim 7, wherein
the light emitter has a first light emitter and a second light emitter,
the light receiver is disposed between the first light emitter and the second light emitter, and
the light blocking wall is disposed between the first light emitter and the light receiver and between the second light emitter and the light receiver.

12. The biological information measurement device according to claim 8, wherein
the light emitter has a first light emitter and a second light emitter,
the light receiver is disposed between the first light emitter and the second light emitter, and
the light blocking wall is disposed between the first light emitter and the light receiver and between the second light emitter and the light receiver.

13. The biological information measurement device according to claim 5, wherein
the light blocking wall surrounds the light receiver viewed from a direction perpendicular to the board.

14. The biological information measurement device according to claim 5, wherein
a tip part of the light blocking wall substantially coincides with a surface of the sealing material.

15. The biological information measurement device according to claim 1, wherein
a surface on which the sealing material has contact with the living body is a curved surface.

16. The biological information measurement device according to claim 2, wherein
a surface on which the sealing material has contact with the living body is a curved surface.

17. The biological information measurement device according to claim 3, wherein
a surface on which the sealing material has contact with the living body is a curved surface.

18. The biological information measurement device according to claim 5, wherein
a surface on which the sealing material has contact with the living body is a curved surface.

19. The biological information measurement device according to claim 1, further comprising:
a signal processor disposed on the second surface, and adapted to process a signal output from the light receiver; and
a housing adapted to house the board,
wherein the housing has a recessed part, and
at least apart of the signal processor is disposed inside the recessed part.

20. The biological information measurement device according to claim 5, further comprising:
an analog front end disposed on the second surface, and adapted to process a signal output from the light receiver; and
a housing adapted to house the board,
wherein the housing has a recessed part, and
at least a part of the analog front end is disposed inside the recessed part.
